(19) United States
(12) Patent Application Publication
(10) Pub. No.: US 2023/0302025 A1
(43) Pub. Date: Sep. 28, 2023

(54) METHODS FOR MAINTAINING MICROVASCULAR INTEGRITY

(71) Applicant: SCISPARC LTD., Tel-Aviv (IL)

(72) Inventors: Ascher SHMULEWITZ, Tel Aviv (IL); Adi ZULOFF-SHANI, Ramat HaSharon (IL); Ephraim BRENER, Rishon LeZion (IL)

(73) Assignee: SCISPARC LTD., Tel-Aviv (IL)

(21) Appl. No.: 18/019,210

(22) PCT Filed: Aug. 2, 2021
(86) PCT No.: PCT/IB2021/057064

§ 371 Date: Feb. 1, 2023

Related U.S. Application Data
(60) Provisional application No. 63/060,245, filed on Aug. 3, 2020.

(51) Int. Cl. A61K 31/00; A61P 9/14

(52) U.S. Cl. CPC A61K 31/658; A61P 9/14

(57) ABSTRACT

The present disclosure provides a method for preventing and/or treating a damage to micro vascular integrity comprising administering to a subject, a composition comprising selected cannabinoid, thereby maintaining the microvascular integrity.

This application claims the benefit of U.S. Provisional Application No. 63/060,245, filed on Aug. 3, 2020, the contents of which are incorporated by reference herein in their entirety.

This disclosure relates to the use of certain cannabinoid-based therapies in preventing and/or treating damage to microvascular integrity, and thus conditions related to injured microvasculature. In particular, the present disclosure relates to the methods of preventing and/or treating relevant disorders with pharmaceutical compositions containing Δ9-tetrahydrocannabinol (THC) in the disclosed compositions.

Microvasculature is made up of the smallest blood vessels in a body. Common examples of microvessels include, but not limited to, arterioles (a small diameter blood vessel that extends and branches out from an artery and leads to capillaries), capillaries (the smallest blood vessels), metarterioles (a vessel that links arterioles and capillaries), venules (a blood vessel that allows deoxygenated blood to return from the capillary beds to the larger blood vessels called veins), and thoroughfare channels (a venous vessel receiving blood directly from capillary beds, and is a tributary to venules).

The main function of the microvasculature is transport of materials. Water and solutes are carried by blood through the microvessels and exchanged, through vessel walls, with the surrounding tissues. This transport function is highly dependent on the architecture of the microvasculature and on the biophysical behavior of blood flowing through it. The hydrodynamic resistance of a microvascular network, which determines the overall blood flow for a given perfusion pressure, depends on the number, size and arrangement of microvessels, the passive and active mechanisms governing their diameters, and on the apparent viscosity of blood flowing in them.

The regulation of tissue perfusion occurs in microcirculation. There, arterioles control the flow of blood to the capillaries. Arterioles contract and relax, varying their diameter and vascular tone, as the vascular smooth muscle responds to diverse stimuli. Distension of the vessels due to increased blood pressure is a fundamental stimulus for muscle contraction in arteriolar walls. As a consequence, microcirculation blood flow remains constant despite of changes in systemic blood pressure. This mechanism is present in all tissues and organs of the human body. In addition, the nervous system participates in the regulation of microcirculation. The sympathetic nervous system activates the smaller arterioles, including terminals. Noradrenaline and adrenaline have effects on alpha and beta adrenergic receptors. Other hormones (catecholamine, renin-angiotensin, vasopressin, and atrial natriuretic peptide) circulate in the bloodstream and can have an effect on the microcirculation causing vasodilation or vasoconstriction. Many hormones and neuropeptides are released together with classical neurotransmitters.

Arterioles respond to metabolic stimuli that are generated in the tissues. When tissue metabolism increases, catabolic products accumulate leading to vasodilation. The endothelium begins to control muscle tone and arteriolar blood flow tissue. Endothelial function in the circulation includes the activation and inactivation of circulating hormones and other plasma constituents. There are also synthesis and secretion of vasodilator and vasoconstrictor substances for modifying the width as necessary. Variations in the flow of blood that circulates by arterioles are capable of responses in endothelium.

Damages to microvasculature are observed in various pathologies, including but not limited to the aging processes, Diabetes Mellitus, Arteriosclerosis, chronic thromboembolic pulmonary hypertension (CTEPH), Portal Atresia (Hepatic Microvascular Dysplasia), nonalcoholic fatty liver disease (NAFLD), Chronic Kidney Disease, Small vessel disease, Polycystic ovarian syndrome, Chronic inflammation, Traumatic Brain Injury (TBI), etc. Microvasculature damages also negatively affect Blood Brain Barrier (BBB). For some said pathologies, the damages to microvasculature precede the development of said pathology and may be a cause or one of the causes leading to such pathology. For some pathologies, microvascular damages progress during or following the development of the pathology.

To diagnose damages to microvasculature, the affected individual's medical history and family history is examined. The tests for damages to microvasculature usually include Stress test with imaging, Coronary angiogram, Positron emission tomography (PET), CT scan or CT angiography (CTA) scan, MRI, and Endothelial dysfunction test.

Treatment of microvascular injuries often depends on the underlying cause of the damage, whether it resulted by high blood pressure, high cholesterol, obesity, diabetes, aging or brain injury. There are no studies about preventing microvascular injuries, and the only recommendation for it, is controlling the disease's major risk factors—high blood pressure, high cholesterol and obesity. Pharmacological treatment for the already present microvascular injuries involves medications to control the narrowing of small blood vessels that could lead to a heart attack and to relieve pain. Some of the agents usually prescribed include Nitroglycerin, Beta blockers, Calcium channel blockers, Statins, Angiotensin-converting enzyme (ACE) inhibitors, Angiotensin II receptor blockers (ARBs), Ranolazine (Ranexa) and Aspirin. Needless to say, that each of these therapies is often also accompanied by undesired side effects. Thus, there is a need to develop a safe and effective therapy that is both prophylactic and therapeutic, and/or can be used as an adjuvant to current therapies.

Cannabis is a genus of flowering plants from order Rosales, family Cannabaceae, which includes three different species, Cannabis sativa, Cannabis indica, and Cannabis ruderalis, which are indigenous to Central and South Asia. Cannabis has long been used for hemp fibre, for seed and seed oils, for medicinal purposes, and well as being a recreational drug. Pharmacologically, Cannabis contains 483 known chemical compounds, including at least 85 different cannabinoids. Cannabinoids, terpenoids, and other compounds are secreted by glandular trichomes that occur most abundantly on the floral calyxes and bracts of female plants.

Cannabinoids are a class of diverse chemical compounds that act on cannabinoid receptors on cells that repress neurotransmitter release in the brain. Cannabinoid receptors are of a class of cell membrane receptors under the G protein-coupled receptor superfamily. As is typical of G protein-coupled receptors, the cannabinoid receptors contain seven transmembrane spanning domains. There are two known subtypes of cannabinoid receptors, termed CB1 and CB2, with mounting evidence of more. The CB1 receptor is expressed mainly in the brain (central nervous system), but also in the lungs, liver and kidneys. The CB2 receptor is expressed mainly in the immune system and in hematopoietic cells. The protein sequences of CB1 and CB2 receptors are about 44% similar.

These compounds include the endocannabinoids (produced naturally in the body by humans and animals, such as Anandamide(AEA) and 2-Arachidonoylglycerol (2-AG)), the phytocannabinoids (found in cannabis and some other plants, such as tetrahydrocannabinol (THC), cannabidiol (CBD) and cannabinol (CBN)), and synthetic cannabinoids (manufactured chemically: JWH-018, JWH-073, CP-47,497, JWH-200, and cannabicyclohexanol).

All classes of phytocannabinoids derive from cannabigerol-type compounds and differ mainly in the way this precursor is cyclized. The classical cannabinoids are derived from their respective 2-carboxylic acids (2-COOH) by decarboxylation (catalyzed by heat, light, or alkaline conditions). Phytocannabinoids (those derived from the Cannabis plant) include but not limited to: tetrahydrocannabinol (THC), tetrahydrocannabinolic acid (THCA), cannabidiol (CBD), cannabinol (CBN), Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM). The main way in which the cannabinoids are differentiated is based on their degree of psychoactivity. For example, CBG, CBC and CBD are not known to be psychologically active agents whereas THC, THCA, CBN and CBDL along with some other cannabinoids are known to have varying degrees of psycho-activity.

The most notable cannabinoid is the phytocannabinoid □9-tetrahydrocannabinol (THC), which is the primary psychoactive component of the cannabis plant. THC has approximately equal affinity for the CB1 and CB2 receptors, and it possesses activities such as an analgesic, psychoactive agent, muscle relaxant, antispasmodic, bronchodilator, neuroprotective, antioxidant and antipruritic agent.

Dronabinol is the International Nonproprietary Name (INN) for a pure isomer of THC, (−)-trans-□9-tetrahydrocannabinol. Synthesized dronabinol is marketed as Marinol. In the United States, Marinol is a Schedule III drug, available by prescription, considered to be non-narcotic and to have a low risk of physical or mental dependence. Marinol has been approved by the U.S. Food and Drug Administration (FDA) in the treatment of anorexia in AIDS patients, as well as for refractory nausea and vomiting of patients undergoing chemotherapy. An analog of dronabinol, nabilone, with therapeutic use as an antiemetic and as an adjunct analgesic for neuropathic pain, is available commercially in Canada under the trade name Cesamet, manufactured by Valeant Pharmaceuticals. Cesamet has also received FDA approval and began marketing in the U.S. in 2006. Nabilone is a Schedule II drug.

Information about the toxicity of THC is primarily based on results from animal studies. The toxicity depends on the route of administration and the laboratory animal. The estimated lethal dose of intravenous dronabinol in humans is 30 mg/kg. The adverse effects of THC are mainly psychoactive. THC intoxication is well established to impair cognitive functioning on an acute basis, including effects on the ability to plan, organize, solve problems, make decisions, and control impulses. Some studies have suggested that cannabis users have a greater risk of developing psychosis than non-users. In addition, chronic use is associated with elevated levels of apolipoprotein C-III (apoC-III). An increase in apoC-III levels induces the development of hypertriglyceridemia.

Cannabidiol (CBD) is a major phytocannabinoid, accounting for up to 40% of the plant's extract in selected cultivars. CBD is considered to have a wider scope of medical applications than tetrahydrocannabinol (THC). An orally-administered liquid containing CBD has received orphan drug status in the US, for use as a treatment for Dravet syndrome, under the brand name Epidiolex. CBD is able to reduce THC induced cognitive impairment and deficits of visuospatial associative memory. CBD also appears to counteract the sleep-inducing effects of THC. Sativex (GW Pharmaceuticals) is the first natural cannabis plant derivative to gain full market approval. Sativex is a mouth spray for multiple sclerosis (MS) derived neuropathic pain, spasticity, overactive bladder, and other symptoms. Each spray delivers a near 1:1 ratio of CBD to THC, with a fixed dose of 2.7 mg THC and 2.5 mg CBD. The endocannabinoid system is an ancient, evolutionarily conserved, and ubiquitous lipid signaling system found in all vertebrates, and which appears to have important regulatory functions throughout the human body. The endocannabinoid system has been implicated in a very broad number of physiological as well as pathophysiological processes including neural development, immune function, inflammation, appetite, metabolism and energy homeostasis, cardiovascular function, digestion, bone development and bone density, synaptic plasticity and learning, pain, reproduction, psychiatric disease, psychomotor behaviour, memory, wake/sleep cycles, and the regulation of stress and emotional state.

The system consists of the cannabinoid 1 and 2 (CB1 and CB2) receptors, the CB receptor ligands N-arachidonoylethanolamine (anandamide or AEA) and 2-arachidonoylglycerol (2-AG) as well as the endocannabinoid-synthesizing and degrading enzymes fatty acid amide hydrolase (FAAH) and monoacylglycerol lipase (MAGL). AEA and 2-AG are considered the primary endogenous mediators of cannabinoid signaling, but other endogenous molecules which exert cannabinoid-like effects have also been described. These other molecules include 2-arachidonoylglycerol ether, N-arachidonoyl dopamine (NADA), virodhamine, N-homo-Q-linolenoylethanolamine (HEA) and N-docosatetraenoylethanolamine (DEA). Molecules such as palmitoylethanolamide (PEA) and oleoylethanolamide (OEA) do not appear to bind to cannabinoid receptors but rather to a specific isozyme belonging to a class of nuclear receptors/transcription factors known as peroxisome proliferator-activated receptors (PPARs)(5). These endocannabinoid-like compounds may, however, potentiate the effect of anandamide by competitive inhibition of FAAH, and/or through direct allosteric effects on other receptors such as the transient receptor potential vanilloid (TRPV1) channel. These types of effects have been generally referred to as the so-called entourage effect.

Endocannabinoids are arachidonic acid derivatives which are synthesized on demand from membrane phospholipid precursors in response to cellular requirements.

Anandamide (N-arachidonoylethanolamine, AEA), one of the major components of endocannabinoid system, is a THC mimmetic. Its effects can be either central, in the brain, or peripheral, in other parts of the body and are mediated primarily by CB1 in the central nervous system, and CB2 in the periphery. However, short half-life due to the action of the enzyme fatty acid amide hydrolase (FAAH), presents a disadvantage for potential therapeutic use.

While there are many anecdotal reports concerning the therapeutic value of cannabis, clinical studies supporting the safety and efficacy of smoked cannabis for therapeutic purposes in a variety of disorders are limited, but slowly increasing in number. Amongst the many therapeutic implications of THC one can find its potential use in palliative care (pain and other distressing symptoms, and the enhancement of quality of life), in treatment of vomiting and nausea, in wasting syndrome (cachexia, e.g., from tissue injury by infection or tumor) and loss of appetite (anorexia) in AIDS and cancer patients, and anorexia nervosa, as well as in Multiple Sclerosis, Amyotrophic Lateral Sclerosis, spinal cord injury, Epilepsy, Arthritides and Musculoskeletal Disorders, Movement disorders, Glaucoma, Asthma, Hypertension, Psychiatric disorders, Alzheimer's disease and dementia, Inflammation, Gastrointestinal system disorders, Anti-neoplastic properties and atherosclerosis.

The current disclosure provides for using cannabinoids in specific dosages and dose ranges to prevent and/or treat microvascular damages and thus address the many pathologies resulted from such damages. In particular, preventing and/or treating microvascular damage may ameliorate the symptoms of Traumatic Brain Injury (TBI) and damages to BBB, Portal Atresia (Hepatic Microvascular Dysplasia), or Chronic inflammation.

### SUMMARY OF THE INVENTION

The present disclosure provides cannabinoid-based compositions and dosage forms for preventing and/or treating damages to microvascular integrity, and thus conditions related injured microvasculature. In particular, the present disclosure relates to the methods of preventing and/or treating relevant disorders with pharmaceutical compositions containing Δ9-tetrahydrocannabinol (THC) in the disclosed compositions.

The present disclosure is based in part on unexpected experimental findings that certain dosage forms of cannabinoids protects small blood vessels from injuries and treat compromised microvasculature, thus maintain microvascular integrity.

The present disclosure provides, in one aspect, a pharmaceutical composition comprising a therapeutically-effective amount of cannabinoid or a salt thereof. In certain embodiments, a pharmaceutical composition comprises more than one cannabinoid.

In certain embodiments, the pharmaceutical composition comprises about 0.01 mg to about 600 mg of at least one cannabinoid or a salt thereof. In certain embodiments, at least one cannabinoid is Δ9-tetrahydrocannabinol (THC). In certain embodiments, the pharmaceutical composition comprises about 0.01 mg, 0.05 mg, 0.1 mg, 0.5 mg, 1 mg, 2.5 mg, 5 mg, or 10 mg THC. Each possibility represents a separate embodiment of the disclosure.

In certain embodiments, the at least one cannabinoid is CBD. In certain embodiments, the pharmaceutical composition comprises about 0.1 mg, 0.5 mg, 1 mg, 10 mg, 50 mg, 100 mg, 300 mg or 600 mg CBD. Each possibility represents a separate embodiment of the disclosure.

In certain embodiments, the pharmaceutical composition comprises about 0.01 to 600 mg of other cannabinoids or salt thereof. In certain embodiments, the other cannabinoids include but are not limited to Phytocannabinoids such as tetrahydrocannabinolic acid (THCA), cannabinol (CBN), Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM). In certain embodiments, the other cannabinoids include but are not limited to Endocannabinoids such as Anandamide(AEA) and 2-Arachidonoylglycerol (2-AG)). In certain embodiments, the other cannabinoids include but are not limited to synthetic cannabinoids such as HU-210, HU-211, HU-308, HU-433, JWH-018, JWH-073, CP-47,497, JWH-200, and cannabicyclohexanol. Each possibility represents a separate embodiment of the disclosure.

In certain embodiments, the cannabinoid-based composition is formulated for systemic administration. In certain embodiments, the pharmaceutical composition is formulated for oral, vaginal, rectal, oral mucosal, nasal, sublingual, inhalational, topical, parenteral, intravenous, intramuscular, or subcutaneous administration. Each possibility represents a separate embodiment of the disclosure. In certain embodiments, the pharmaceutical composition is formulated for oral, topical, or oral mucosal/buccal administration. In certain embodiments, the pharmaceutical composition is formulated as a solution or as a suppository.

Embodiments of the present disclosure further provide a dosage unit comprising the pharmaceutical composition described above.

The present disclosure further provides, in another aspect, a cannabinoid-based composition as described above for use in a method for preventing and/or treating the damage to microvascular integrity. In certain embodiments, the damage to microvascular integrity is due to the aging processes, Diabetes Mellitus, Arteriosclerosis, chronic thromboembolic pulmonary hypertension (CTEPH), Portal Atresia (Hepatic Microvascular Dysplasia), nonalcoholic fatty liver disease (NAFLD), Chronic Kidney Disease, Small vessel disease, Polycystic ovarian syndrome, Chronic inflammation, Traumatic Brain Injury (TBI), etc. In certain embodiments, the cannabinoid-based composition is used in the manufacture of a medicament for preventing and/or treating the damage to microvascular integrity in a subject in need of such treatment.

The present disclosure further provides, in another aspect, a method of preventing and/or treating the damage to microvascular integrity comprising administering to a subject in need thereof a therapeutically-effective amount of at least one cannabinoid or salt thereof.

In certain embodiments, the therapeutically-effective amount of the cannabinoid or a salt thereof is from about 0.01 mg to about 600 mg. In certain embodiments, the at least one cannabinoid is THC. In certain embodiments, the said amount of THC is about 0.01 mg, 0.05 mg, 0.1 mg, 0.5 mg, 1 mg, 2.5 mg, 5 mg, or 10 mg THC. In certain embodiments, the at least one cannabinoid is CBD. In certain embodiments, the said amount of CBD is about 0.1 mg, 0.5 mg, 1 mg, 10 mg, 50 mg, 100 mg, 300 mg or 600 mg CBD. Each possibility represents a separate embodiment of the disclosure.

In certain embodiments, the cannabinoids are formulated for systemic administration. In certain embodiments, the cannabinoids are formulated for oral, vaginal, rectal, oral mucosal, nasal, sublingual, inhalational, topical, parenteral, intravenous, intramuscular, or subcutaneous administration. Each possibility represents a separate embodiment of the disclosure. In certain embodiments, the cannabinoids are formulated for oral, topical, or oral mucosal/buccal administration. In certain embodiments, the cannabinoids are formulated as a solution or as a suppository.

In certain embodiments, the cannabinoids as described in the method above are orally administered. In certain embodiments, the cannabinoids are administered once daily. In certain embodiments, the cannabinoids are administered more than once daily.

Other objects, features, and advantages of the present disclosure will become clear from the following description.

### BRIEF DESCRIPTION OF THE DRAWINGS

In order to provide an understanding of non-limiting embodiments of the disclosure, reference is made to the appended drawings, which are not necessarily drawn to scale, and in which reference numerals refer to components of exemplary embodiments of the disclosure. The drawings are exemplary only and should not be construed as limiting.

FIG. 1A is an analysis of blood-brain barrier permeability using contrast-enhanced MRI via Permeability maps calculated from CE-MRI (T1 sequence).

FIG. 1B is an analysis of blood-brain barrier permeability using contrast-enhanced MRI showing cumulative distribution function of contrast enhancement slope following injection of the non-permeable Gd-based contrast agent. Note the right shift of the distribution in animals exposed to TBI, indicating larger number of voxels with positive voxels (i.e. higher permeability). Pathological BBB was defined as a slope value above the 95th percentile of controls.

FIG. 1C is an analysis of blood-brain barrier permeability using contrast-enhanced MRI showing percent (%) of brain volume with pathological voxels is increased following TBI in sensitive and resilient rats.

FIG. 1D is is an analysis of blood-brain barrier permeability using contrast-enhanced MRI showing percent (%) of brain volume with pathological voxels is increased following TBI in THC-treated and control rats. Moderate decrease is observed in the repeated THC-treated group (not significant). Note that repeated THC-treated animals were not different compared to controls. * p<0.05; ** p<0.01; *p<0.001.

FIG. 2A shows blood-brain barrier permeability is reduced in repeated THC-treated animals via the open window method. The open window method allows a direct visualization of microvasculature anatomy and function, while recording brain activity.

FIG. 2B shows blood-brain barrier permeability is reduced in repeated THC-treated animals via fluorescent angiography which permits permeability measurements.

FIG. 2C shows blood-brain barrier permeability is reduced in repeated THC-treated animals via fluorescent angiography which permits permeability measurements.

FIG. 3A shows the use of paroxysmal slow wave events (PSWEs) as a novel biomarker for brain dysfunction where PSWEs were detected as distinct, transient EEG events characterizing a dysfunctional cortex. PSWEs were defined as EEG slowing (median frequency <6 Hz) for at least 5 consecutive seconds).

FIG. 3B shows the use of paroxysmal slow wave events (PSWEs) as a novel biomarker for brain dysfunction where the number of PSWEs was counted during ECoG recordings from controls and treated rats. Note the significant increase in the number of PSWEs in saline” and THC1-treated rats, while repeated administration of THC resulted in a lower occurrence. *** p<0.001 ** p<0.01.

### DETAILED DESCRIPTION OF THE INVENTION

The present disclosure further provides methods for the use of cannabinoid-based compositions and dosage forms in treating diseases, disorders, and conditions related damage to microvascular integrity.

The cannabinoid-based compositions of the disclosed embodiments provide an improved therapeutic entity compared to current therapies, exhibiting prophylactic and therapeutic activity, while been devoid of serious adverse events usually associated with common medications addressing microvascular damages. The embodiments described herein are based on the discovery that cannabinoids at defined doses act to protect microvasculature from damages and/or repair microvascular injuries.

The present disclosure provides, in one aspect, a cannabinoid-based composition comprising at least one cannabinoid or a salt thereof.

As used herein, a “pharmaceutical composition” refers to a preparation of the active agents described herein with other chemical components such as physiologically suitable carriers and excipients. The purpose of a pharmaceutical composition is to facilitate administration of a compound to an organism. As used herein, the phrase “pharmaceutically acceptable carrier” refers to a carrier, an excipient, or a diluent that does not cause significant irritation to an organism and does not abrogate the biological activity and properties of the administered compound. An adjuvant is included under these phrases.

The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

The term “derivative” as used herein means a compound whose core structure is the same as, or closely resembles that of a reference compound, but which has a chemical or physical modification, such as different or additional side groups.

The term “carrier” as used herein refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E.W. Martin, 18th Edition.

The phrase “pharmaceutically acceptable” as used herein refers to molecular entities and compositions that are physiologically tolerable and do not typically produce an allergic or similar toxicity when administered to an individual. Preferably, and particularly where a formulation is used in humans, the term “pharmaceutically acceptable” may mean approved by a regulatory agency (for example, the U.S. Food and Drug Agency) or listed in a generally recognized pharmacopeia for use in animals (e.g., the U.S. Pharmacopeia).

The term “cannabinoid” as used herein generally refers to one of a class of diverse chemical compounds that acts on cannabinoid receptors in cells that alter neurotransmitter release in the brain. These include the endocannabinoids (produced naturally in the body by animals), the phytocannabinoids (found in cannabis and some other plants), and synthetic cannabinoids (manufactured artificially and encompass a variety of distinct chemical classes: the classical cannabinoids structurally related to THC, the nonclassical cannabinoids (cannabimimetics) including the aminoalkylindoles, 1,5-diarylpyrazoles, quinolines, and arylsulfonamides as well as eicosanoids related to endocannabinoids).

The term “salt” as used herein refers to any form of an active ingredient in which the active ingredient assumes an ionic form and is coupled to a counter ion (a cation or anion) or is in solution. This also includes complexes of the active ingredient with other molecules and ions, in particular complexes which are complexed by ion interaction.

The present disclosure further provides, in another aspect, a dosage unit comprising or consisting of any one of the pharmaceutical compositions described above. Techniques for formulation and administration of drugs are well known in the art, and may be found, e.g. in “Remington's Pharmaceutical Sciences,” Mack Publishing Co., Easton, Pa.

The compositions of the present disclosure may be manufactured by processes well known in the art, e.g., by means of conventional mixing, dissolving, granulating, dragee-making, levigating, emulsifying, encapsulating, entrapping, or lyophilizing processes.

The compositions for use in accordance with the present disclosure may be formulated in a conventional manner using one or more physiologically acceptable carriers comprising excipients and auxiliaries, which facilitate processing of the active ingredients into preparations that can be used pharmaceutically. Proper formulation is dependent upon the route of administration chosen.

For topical application, the active ingredients of the composition may be formulated in cremes, ointments, solutions, patches, sprays, lotions, liniments, varnishes, solid preparations such as silicone sheets, and the like.

The term “topical” as used herein refers to the application of a disclosed composition directly onto at least a portion/region of a subject's skin (human's or non-human's skin) to achieve a desired effect, for example, treating dermatological diseases as described herein.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

The term “mucosal administration” relates to delivery of a composition to a mucous membrane, such as the buccal or labial mucosa or the mucosa of the respiratory tract, such as the nasal mucosa.

For oral administration, the composition can be formulated readily by combining the active compounds with acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose (HPMC), and sodium carbomethylcellulose (CMC); and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The term “oral administration” refers to any method of administration in which an active agent can be administered by swallowing, chewing, sucking, or drinking an oral dosage form. Examples of solid dosage forms include conventional tablets, multi-layer tablets, capsules, caplets, etc., which do not substantially release the drug in the mouth or in the oral cavity.

Dragee cores are provided with suitable coatings. For this purpose, concentrated sugar solutions may be used which may optionally contain gum arabic, talc, polyvinyl pyrrolidone, CARBOPOL gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include stiff or soft, sealed capsules made of gelatin and a plasticizer, such as glycerol or sorbitol. The capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal and sublingual administration, the compositions may take the form of tablets or lozenges formulated in a conventional manner or in adhesive carriers.

The composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulation agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethylcellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The present compositions can also be delivered using an in situ formed depot (ISFD). Examples of in situ formed depots include semi-solid polymers which can be injected as a melt and form a depot upon cooling to body temperature. The requirements for such ISFD include low melting or glass transition temperatures in the range of 25-658° C. and an intrinsic viscosity in the range of 0.05-0.8 dl/g. Below the viscosity threshold of 0.05 dl/g no delayed diffusion could be observed, whereas above 0.8 dl/g the ISFD was no longer injectable using a needle. At temperatures above 378° C. but below 658° C. these polymers behave like viscous fluids which solidify to highly viscous depots. Drugs are incorporated into the molten polymer by mixing without the application of solvents. Thermoplastic pastes (TP) can be used to generate a subcutaneous drug reservoir from which diffusion occurs into the systemic circulation.

In situ cross-linked polymer systems utilize a cross-linked polymer network to control the diffusion of macromolecules over a prolonged period of time. Use of in situ cross-linking implants necessitates protection of the bioactive agents during the cross-linking reaction. This could be achieved by encapsulation into fast degrading gelatin microparticles.

An ISFD can also be based on polymer precipitation. A water-insoluble and biodegradable polymer is dissolved in a biocompatible organic solvent to which a drug is added forming a solution or suspension after mixing. When this formulation is injected into the body the water miscible organic solvent dissipates and water penetrates the organic phase. This leads to phase separation and precipitation of the polymer forming a depot at the site of injection. One example of such a system is ATRIGELE.

Thermally induced gelling systems can also be used as ISFDs. Numerous polymers show abrupt changes in solubility as a function of environmental temperature. The prototype of a thermosensitive polymer is poly(N-isopropyl acryl amide), poly-NIPAAM, which exhibits a rather sharp lower critical solution temperature.

Thermoplastic pastes such as the new generation of poly(ortho esters) developed by AP Pharma can also be used for depot drug delivery. Such pastes include polymers that are semi-solid at room temperature, hence heating for drug incorporation and injection is no longer necessary. Injection is possible through needles no larger than 22 gauge. The drug can be mixed into the systems in a dry and, therefore, stabilized state. Shrinkage or swelling upon injection is thought to be marginal and, therefore, the initial drug burst is expected to be lower than in the other types of ISFD. An additional advantage is afforded by the self-catalyzed degradation by surface erosion.

The compositions of the present disclosure can also be delivered from medical devices, such as orthopedic implants, contact lenses, micro needle arrays, patches and the like.

Sustained-release (SR), extended-release (ER, XR, or XL), time-release or timed-release, controlled-release (CR), or continuous-release (CR) pills are tablets or capsules formulated to dissolve slowly and release a drug over time. Sustained-release tablets are formulated so that the active ingredient is embedded in a matrix of insoluble substance (e.g. acrylics, polysaccharides, etc.) such that the dissolving drug diffuses out through the holes in the matrix. In some SR formulations the matrix physically swells up to form a gel, so that the drug has first to dissolve in matrix, then exit through the outer surface. The difference between controlled release and sustained release is that controlled release is perfectly zero order release. That is, the drug releases with time irrespective of concentration. On the other hand, sustained release implies slow release of the drug over a time period. It may or may not be controlled release.

Pharmaceutical compositions suitable for use in the context of the present disclosure include compositions wherein the active ingredients are contained in an amount effective to achieve the intended purpose. More specifically, a “therapeutically effective amount” means an amount of active ingredients effective to prevent, alleviate, or ameliorate symptoms or side effects of a disease or disorder, or prolong the survival of the subject being treated. Determination of a therapeutically effective amount is well within the capability of those skilled in the art, especially in light of the detailed disclosure provided herein.

For any preparation used in the methods of the disclosure, the dosage or the therapeutically effective amount can be estimated initially from in vitro and cell culture assays. For example, a dose can be formulated in animal models to achieve a desired concentration or titer. Such information can be used to more accurately determine useful doses in humans.

The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

The dosage of cannabinoid, such as THC or CBD, within the claimed composition when taken orally may be ranged from 0.01 mg to about 600 mg cannabinoid per 50 kg subject daily, within maximal single dose of 600 mg or 12 mg/kg per 24 hours.

Continuous daily dosing may not be required; a therapeutic regimen may require cycles, during which time a drug is not administered, or therapy may be provided on an as-needed basis during periods of acute disease worsening.

The present disclosure further provides, in another aspect, a cannabinoid-based composition described above, or a dosage unit described above, for use in a method for preventing and/or treating a condition, resulting from microvascular injuries or damages.

Microvascular injury is defined as a damage to the finer blood vessels in the body, including the capillaries. The source of the damage to microvascular integrity varies and can be caused without limitations by aging processes, Diabetes Mellitus, Arteriosclerosis, chronic thromboembolic pulmonary hypertension (CTEPH), Portal Atresia (Hepatic Microvascular Dysplasia), nonalcoholic fatty liver disease (NAFLD), Chronic Kidney Disease, Small vessel disease, Polycystic ovarian syndrome, Chronic inflammation, Traumatic Brain Injury (TBI), etc. For example, the microvascular complications of diabetes such as neuropathy can lead to loss of sensation and the development of foot ulcers, while loss of microvascular integrity during TBI can result in cerebral hypoxia, reduced cerebral perfusion pressure and ischemia.

The term “preventing” as used herein, includes, but is not limited to, any one or more of the following: keeping from happening or continuing, averting, avoiding, blocking, halting, countering, hampering, arresting one or more symptoms or side effects of the diseases or conditions of the disclosed embodiments.

The term “treating” as used herein, includes, but is not limited to, any one or more of the following: abrogating, ameliorating, inhibiting, attenuating, blocking, suppressing, reducing, delaying, halting, alleviating or preventing one or more symptoms or side effects of the diseases or conditions of the disclosed embodiments.

The present disclosure further provides, in another aspect, a pharmaceutical composition described above, or a dosage unit described above, for use in a method for preventing and/or treating microvascular injury.

The present disclosure further provides, in another aspect, a method for preventing and/or treating a microvascular injury in a human subject in need thereof, the method comprising the step of administering to the subject a therapeutically-effective amount of a pharmaceutical composition comprising at least one cannabinoid or a salt thereof, thereby preventing and/or treating a microvascular injury.

Dosage escalation may or may not be required; a therapeutic regimen may require reduction in medication dosage.

Toxicity and therapeutic efficacy of the active ingredients described herein can be determined by standard pharmaceutical in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (Fingl, 1975)

Depending on the severity and responsiveness of the condition to be treated, dosing can be of a single or a plurality of administrations, with course of treatment lasting from several days to several weeks, or until cure is affected or diminution of the disease state is achieved.

Suitable routes of administration may, for example, include oral, rectal, vaginal, topical, nasal, trans-nasal, transmucosal, intestinal, or parenteral delivery, including intramuscular, subcutaneous, and intramedullary injections, as well as intrathecal, direct intraventricular, intravenous, intraperitoneal, intranasal, intraocular injections or by means of inhalation or aspiration (smoking). Alternately, the pharmaceutical composition may be administered locally, rather than in a systemic manner, for example, via injection of the pharmaceutical composition directly into a tissue region of a patient.

In certain embodiments, cannabinoids are orally administered. In certain embodiments, cannabinoids are administered via oral mucosal route. In certain embodiments, cannabinoids are administered via topical route. In certain embodiments, cannabinoids are daily administered. In certain embodiments.

Compositions of the present disclosure may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the disclosure formulated in a pharmaceutically acceptable carrier may also be prepared, placed in an appropriate container, and labeled for treatment of an indicated inflammatory disorder, as further detailed above.

The foregoing description of the specific embodiments will so fully reveal the general nature of the compositions and methods that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

The following examples are presented in order to more fully illustrate some embodiments of the disclosure. They should, in no way be construed, however, as limiting the broad scope of the disclosure. One skilled in the art can readily devise many variations and modifications of the principles disclosed herein without departing from the scope of the disclosure.

Non-limiting embodiments of the present disclosure will be described more fully hereinafter. The disclosed subject matter may, however, be embodied in many different forms and should not be construed as limited to the embodiments set forth herein. Rather, these embodiments are provided so that this disclosure will be thorough and complete, and will fully convey the scope of the disclosure to those skilled in the art. As used in this specification and the claims, the singular forms “a,” “an,” and “the” include plural referents unless the context clearly dictates otherwise.

### EXAMPLES

### Example 1: Evaluation of the Selected Doses of THC Effects as a Therapeutic in Mild Traumatic Brain Injury and Brain Microvasculature Integrity in Rat Model

Complicated mild traumatic brain injury (mTBI) is common and may follow with persistent neurological sequala. When repeated, mild TBI increases the risk to neurodegenerative disorders and even death. The goal of the present study was to test the effect of ultra-low dose of THC in a model of mild, repeated brain injury with neurological complications.

We induced mTBI in 8-week-old male Sprague-Dawley rats. A single impact was associated with transient neurological deficits with full recovery at 24 hours after the impact, with no apparent intracerebral injury in routine MRI examination. Upon repeated injury, 60% of the animals showed a significant neurological deterioration, including paresis, seizures and weight loss. Animals were randomized into three treatment groups: a single dose of THC (0.002 mg/kg), a repeated dose after each TBI (up to 5 daily impacts), and a vehicle group, injected with either a single or repeated dose of the vehicle solution. All experiments and data analysis were performed blindly by researchers blind to treatment.

For each experimental group (17-18 rats) we analyzed neurological scoring and weight loss. In sub-groups of animals MRI was performed for structural anatomy. Contrast-enhanced (CE) MRI and intravital microscopy were performed for vascular integrity (BBB permeability).

All animal procedures were approved by the Dalhousie University Committee on Laboratory Animals. Animals were double-housed in a reverse 12:12 light:dark cycle with food and water available ad libitum. 8-week-old male Sprague-Dawley rats (Charles River, Quebec, Canada—strain 001) were cared for in accordance with the Guide for Care and Use of Experimental Animals of the Canadian Council on Animal Care.

### Methods

A closed-head, weight drop model at a height of 1 m, and an imact weight of 500 g was used to produce a mild brain injury. All animals were behaviorally tested 30 minutes prior each impact. All rmTBI animals were administered 2 L/min O2 and 3.5% isoflurane for 3 minutes (or until no pedal reflex remained) prior to each impact. Animals were given one impact per day for five consecutive days, for a total of maximum five impacts, unless deteriorated neurologically (see below). Recovery after each impact was filmed for off-line analysis of post-traumatic seizures, time to gain full recovery etc. Control, non-traumatized, animals were treated similarly with daily 2 L/min 02 and 3.5% isoflurane (3 minutes, or until no pedal reflex remained) but were not impacted.

Treatment phase: Ten minutes after the trauma, all animals were given an i.p. injection that included: A single THC treatment following the first impact, a single injection of the vehicle solution, repeated injections of THC (injection after each impact), or repeated injections of the vehicle solution. THC injections were prepared in a vehicle solution of 1:1:18 ethanol:cremaphor:saline at a dose of 0.002 mg/kg. Injection volume of 1 mL/kg was used.

Electrocorticographic recordings: Electrodes and wireless transmitters were implanted as described (Bar-Klein, 2017) 3 weeks after the first impact. Briefly, in a stereotaxic frame and 0.8-2% isoflurane anesthesia, the skull was exposed and holes were drilled for screws insertion (0.5 mm rostral or 3.5 mm caudal and 1 mm lateral to each side (4 screws, 2 in each hemisphere). A wireless transmitter (Data Science International, Saint Paul, MN, US) was placed in a dorsal subcutaneous pocket, and its leads were connected to the skull screws. Connections were isolated and fixed by bone cement such that one ECoG channel was associated with each hemisphere. Continuous, bichannel ECoG (sampling rate, 1000 Hz) was recorded wirelessly from freely moving animals in the home cage for 2 weeks.

Intravital microscopy: For direct visualization of cortical microvasculature, the open window method was used as previously described (Prager, 2010; Schoknecht, 2016). Briefly, 5-8 days after the first injury rats were deeply anesthetized using a mixture of ketamine (100 mg/ml, 0.08 ml/100 g) and xylazine (20 mg/ml, 0.06 ml/100 g). The tail vein was cannulated and the animal was placed in a stereotactic frame. Body temperature was maintained at 37.0±0.5° C. (RWD Life Science CO, LTD), and oxygen-enriched air (99.5%) was provided through a nose tube. A craniotomy bone window was drilled over the motor-somatosensory cortex of the right hemisphere (4 mm caudal, 2 mm frontal, 5 mm lateral to bregma), and the dura was carefully removed.

A bone-cement ring was built around the bone window, and the cortex was continuously perfused with artificial cerebrospinal fluid (aCSF, pH 7.4) containing (in mM): 129 NaCl, 21 NaHCO₃, 1.25 NaH2PO4, 1.8 MgSO4, 1.6 CaCl2), 3 KCl, and 10 glucose. Fluorescent angiography was performed for quantitative assessment of BBB integrity by injecting the BBB-non-permeable dye NaFlu (MW=376 Da, Novartis, 1 mg/ml in saline, Ex/Em, 470/525 nm, respectively). Images were acquired from the surface of the cortex (1530 frames, 5 Hz) using the EMCCD camera. For quantification, images were pre-processed (image resizing and registration) and segmented into arteriolar, venous and extravascular compartments. The mean area under the curve of the extravascular compartment was used as a measure of BBB permeability.

Magnetic Resonance Imaging: Scanning protocol and analysis as reported (Bar-Klein et al., 2017; Tagge et al., 2017). Briefly, dynamic contrast-enhanced MRI (DCE-MRI) was performed 1 week and 1 month following the rmTBI protocol using a 3 Tesla Agilent system under isoflurane anesthesia (1-2%) with a constant oxygen flow (99%, 11/h). Breathing was monitored continuously during imaging using a respiration monitor. Scanning protocols included: (i) standard T2-weighted fast spin echo sequence (repetition time: 2500 ms; echo time: 64 ms; echo train length of 16, echo spacing 8 ms; 46 averages; 128×128 data matrix, resulting in 0.297 mm in-plane resolution and a slice thickness of 1 mm; acquisition time: 15.3 min); acquired prior to gadolinium injections (ii) two balanced steady state free precession (bSSFP) 3D T1-weighted scans (repetition time: 8 ms; echo time: 4 ms; 4 frequencies, 10 s segment delay; 176×160×146 data matrix, resulting in 0.25 mm in-plane resolution, and 0.3 mm in the 2nd phase dimension; acquisition time: 13.1 min), one before and one approximately 25 minutes after the injection of the gadolinium based tracer (multihance; gadobenate dimeglumine, IV, ˜211.6 mg/rat; (iii) ten transverse T1-weighted gradient-echo classic scans (repetition time: 6.03 ms; echo time: 2.98 ms; flip angle: 20o; 20 averages; 108×108 data matrix, resulting in 0.352 mm in-plane resolution and a slice thickness of 1.2 mm; acquisition time: 3 min) were performed, one immediately before and eight immediately following the injection of the multihance (gadobenate dimeglumine, IV, ˜211.6 mg/rat). One final transverse T1-weighted scan was acquired approximately 40 min post-injection as a final timepoint. Analysis was performed using in-house Matlab scripts. Pre-processing included registration, extracting brain volume and creating brain mask objects. To visualize BBB integrity (represented as slope images), we used the linear dynamic method by fitting a linear curve to the dynamic scan intensities of the eight consecutive post-contrast T1 scans. That is, a signal s(t) is fitted to a linear curve such that: s(t)=A H t+B, where the slope (A) is the rate of wash-in or wash-out of the contrast agent from the brain. Additionally, for quantitative comparisons in BBB dysfunction, a “pathological” voxel threshold was set as any slope value exceeding the 95% percentile slope value of no-impact, anesthesia control animals (n=6).

### Results

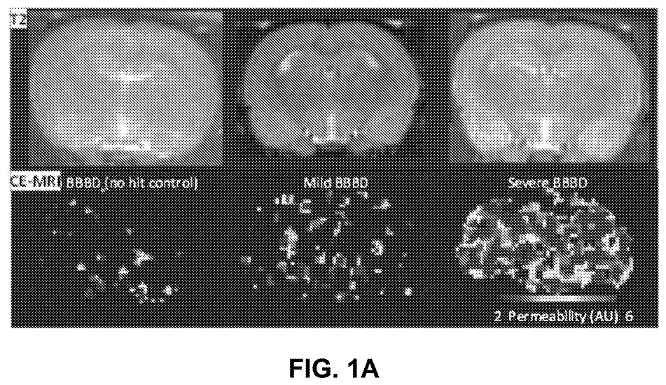

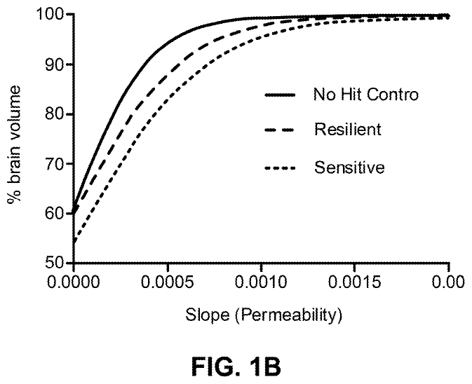

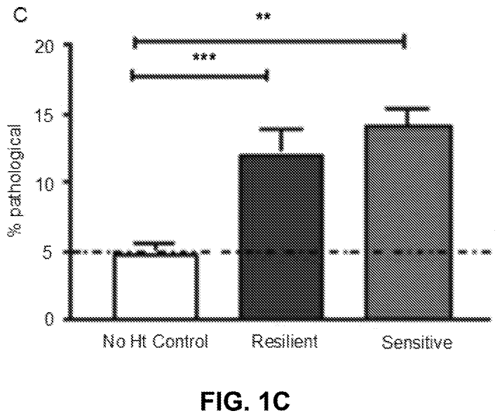

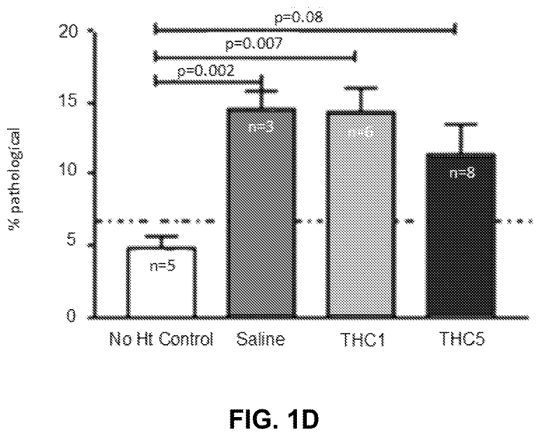

Effect of THC on microvascular integrity: Brain magnetic resonance imaging (MRI) was performed one week and one month after the first injury. As expected from a model designed to reflect a mild TBI, structural MRI showed no anatomical lesion. However, consistent with previous findings in concussed football players (Weissberg et al., JAMA Neurology, 2015), contrast-enhanced MRI shows brain accumulation of the contrast agent, indicating a leaky blood-brain barrier (BBB). We calculated the threshold representing maximal normal brain permeability as that observed in 95% of brain volume of healthy non-injured control rats. We next calculated for each brain the % of brain volume with permeability above threshold, termed as brain with “pathological BBB”. Cumulative distribution function of permeability values showed shift to the right in injured animals (FIG. 1), indicating higher permeability values in traumatized animals, specifically in the sensitive group. Percentage of brain volume with BBB dysfunction was significantly higher in injured rats compared to controls. Animals treated daily with THC, showed a moderate decrease in brain volume with damaged BBB.

Plotting the distribution of all measured neurological scores from mTBI-exposed animals (n=30), shows a clear single distribution of the data on day 1. In contrast, on day 4 (following 3 repeated impacts) a clear bimodal distribution is seen. Accordingly, we considered rats which at any day during the follow-up scored <6 in the neurological (DUCS) score as a “sensitive” rat, while animals that always scored >=6, as “resilient”.

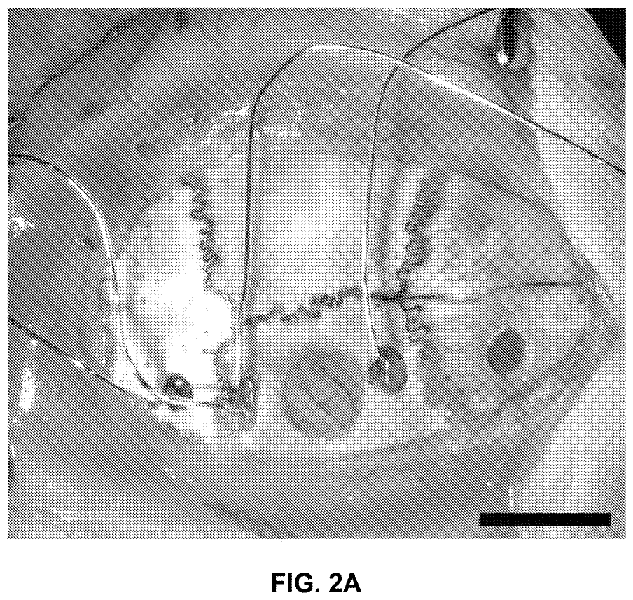

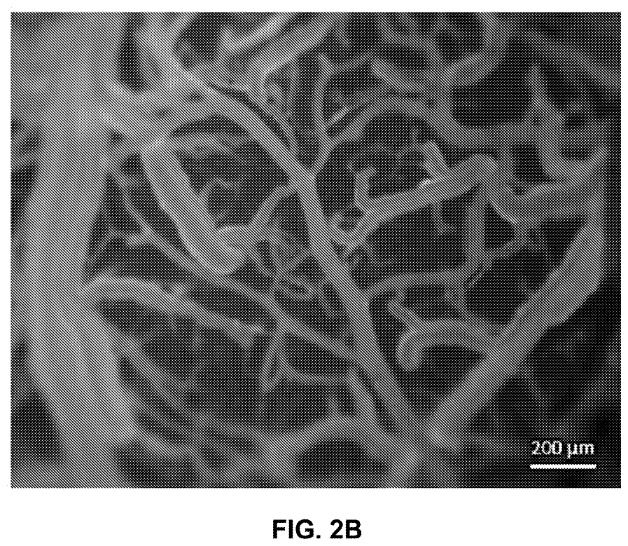

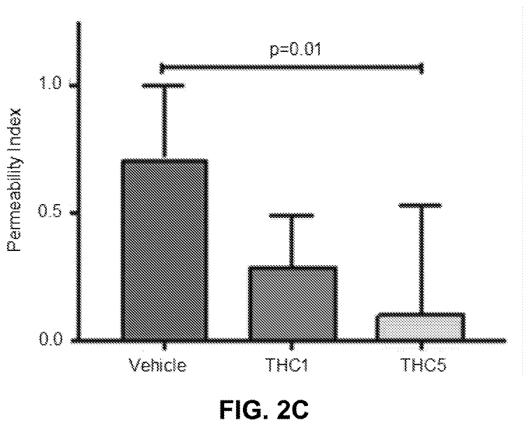

Intravital microscopy: To confirm MRI results suggesting THC protecting effect on microvascular integrity, we visualized directly small cortical vessels 6-8 days after the first injury, using intravital microscopy in the open window approach. BBB permeability was measured following the peripheral injection of a fluorescent agent (AKA fluorescent angiography) as we previously reported (Prager et al., 2010; Schoknecht et al., 2016; Vazana et al., 2016; and see FIG. 2). Consistent with our MRI data, cortical microvascular permeability in repeated-THC treated animals was lower compared to vehicle-injected controls, suggesting preservation of microvascular integrity.

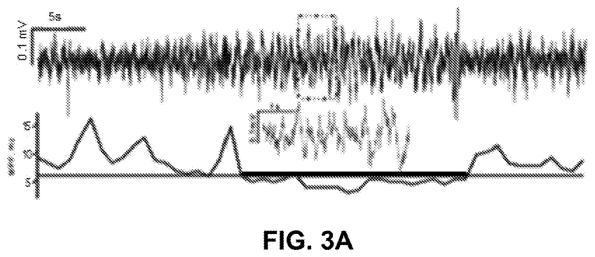

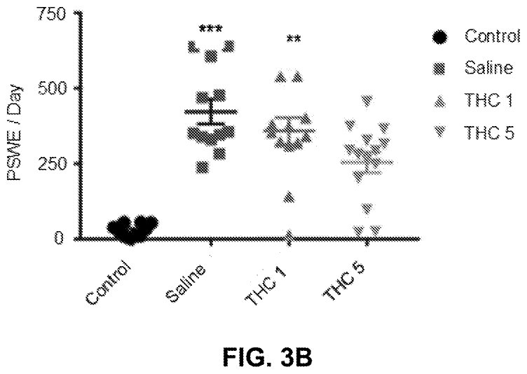

Effect of THC on EEG biomarkers of brain injury: Electrocorticography (ECoG) was recorded 3-4 weeks following injury (see Methods). The following outcome measures for brain injury were quantitatively, objectively and blindly assessed: “paroxysmal slow wave events” (PSWEs): These are transient, paroxysmal cortical slowing recently shown to characterize the injured, and likely BBB-deprived cerebral cortex. The algorithm quantify the number of events (time windows in which median frequency is dropped <5 Hz for at least 6 seconds) in each recording. As can be seen in FIG. 3, while control animals show very few events, TBI-exposed rats show a large number of PSWEs/day. Interestingly, and consistent with MRI and imaging data described above, rats exposed to repeated treatment with THC had mild reduction in the number of PSWEs and a larger variance (FIG. 3).

### Summary

Electrocorticographic recordings were performed for the detection of spontaneous seizures. The daily injection of THC reduced the risk for neurological complications from 60% to 35%. Data from both CE-MRI as well as intravital microscopy suggest a mild protective and therapeutic effect of repeated treatment with THC on trauma-induced BBB breakdown.

### Example 2: Evaluating the Effect of THC and CBD on Changes of Blood Flow in Retina, Choroid, and Outer Choroid in Rats Model of Proliferative Diabetic Retinopathy (PDR)

Diabetic retinopathy (DR) is one of the most common complications of diabetes and is a leading cause of blindness in people of the working age in Western countries. A major pathology of DR is microvascular complications such as non-perfused vessels, microaneurysms, dot/blot hemorrhages, cotton-wool spots, venous beading, vascular loops, vascular leakage and neovascularization.

The objective of the study is to assess the effects of THC and/or CBD in treating microvascular damages derived from the development of DR in streptozotocin (STZ) induced rat model.

Study variables and endpoints: mortality and morbidity are measured once a day. Clinical observations are made daily, with special attention given for signs of limping, infection, or edema in the injected subject. Body weight measurements are performed throughout the study, specifically upon arrival, before study initiation, and once a week thereafter upon study termination.

The principle of the study is based on the knowledge that injection of STZ induces irreversible changes in pancreas leading to the development of Diabetes. Male Wistar rats weighing 150-220 g are used. STZ (60 mg/kg) is injected intravenously. The serum insulin values decrease up to 4 times, after six to eight hours of injection, resulting in a hypoglycemic phase after persistent hyperglycemia. Diabetic symptoms severity and onset depend on the dose of STZ. After the dose of 60 mg/kg i.v., symptoms occur already after 24-48 h with hyperglycemia up to 800 mg %, glucosuria and ketonemia.

Histologically degranulation of the beta cells is seen. After 10-14 days a steady state is reached allowing using the animals for pharmacological tests.

Animal Handling: Animal handling is performed according to guidelines of the National Institute of Health (NIH) and the Association for Assessment and Accreditation of Laboratory Animal Care (AAALAC), and Pharmaseed's SOPs. Animals are housed in individual ventilated cages (IVC) (maximum 3 rats/cage) measuring 42.5×26.5×18.5 cm, with stainless steel top grill facilitating pelleted food and drinking water in plastic bottles; bedding: individual paper bedding for assessing poliuria. Bedding material is changed along with the cage once daily. Animals are fed ad libitum a commercial rodent diet (Teklad Certified Global 18% Protein Diet, Harlan cat #2018SC). Animals have free access to sterilized and acidified drinking water (pH between 2.5 and 3.5) obtained from the municipality supply and treated according to Pharmaseed's SOP No. 214: “Water system.”

Study Design: Rats are randomly allocated to cages on the day of reception. Allocation and randomization to relevant groups is done on Day 0. Animals are allocated to seven treatment groups as indicated below:
OM Control (naive)
1M Control (STZ injected, non-diabetic)
2M Contol (STZ injected, diabetic (DM))
3M DM THC treated 0.002 mg/kg
4M DM THC treated 0.02 mg/kg
5M DM CBD treated 0.02 mg/kg
6M DM CBD treated 0.2 mg/kg

Following STZ injection, animals are monitored daily on blood glucose levels (BG), polyuria, and vital signs. Animals that does not develop Diabetes (BG >250) are allocated to group 1M. Five days following the development of Diabetes animals begin treatment with either THC or CBD in accordance with listed above for the duration of additional five days.

Five days following the development of Diabetes animals begin treatment with either THC or CBD in accordance with listed above for the duration of additional five days.

Morbidity and mortality checks are performed once a day. The animals, which will be humanely killed during the test, are considered for the interpretation of test results as animals that died during the test. In case of mortality before study scheduled termination, gross pathology evaluation is performed as close as possible to the time of death. The time of death will be recorded as precisely as possible.

The animals are observed for toxic/adverse symptoms daily, until study termination. Body weight is recorded upon arrival, before STZ injection, two days after STZ injection, and once weekly thereafter according to Pharmaseed's SOP No. 010 “Weighing Laboratory Animals.”

At 2, 3 months after the injection of STZ, the rats were anesthetized by sodium pentobarbital (40 mg/kg, i.p.), the blood samples were taken from the abdominal aorta, and the eyes were removed immediately.

Retinal immunfluorescence staining: The retinas are incubated with 4% paraformaldehyde solution overnight in 4° C., and then are blocked in blocking buffer (5% BSA, 0.5% triton X-100 in PBS) for 1-3 h at room temperature, and then are incubated with the CD31 antibody for 1d or 2d at 4° C., and then washed with washing buffer (0.5% triton X-100 in PBS) for every 20 min. After washing 6 times, the retinas are incubated with FITC-conjugated anti-Rat IgG antibody for 2 h. After washing 6 times again, the retinas are placed on a slide glass, mounted in gelatin, covered with a cover slip, and pictured under the fluorescence microscopy. The quantitative of the vessels is counted as described method (Huang CX 2006). Firstly, two lines, which constitute a cross, are drawn in the central on the pictures. Then, the number of vessels cross these two lines is counted.

Histological assessment: The retina tissues were isolated from the normal rats and diabetic rats, and then fixed in 4% paraformaldehyde solution. Samples were subsequently sectioned (5 micromoL/L), stained with haematoxylin and eosin and examined under the microscopy.

1. A method for preventing and/or treating damage to microvascular integrity, comprising administering to a subject in need thereof a composition comprising one or more cannabinoids, thereby preventing and/or treating the damage to microvascular integrity.

2. The method of claim 1, wherein the damage to microvascular integrity is due to the aging process, Diabetes Mellitus, Arteriosclerosis, chronic thromboembolic pulmonary hypertension (CTEPH), Portal Atresia (Hepatic Microvascular Dysplasia), nonalcoholic fatty liver disease (NAFLD), Chronic Kidney Disease, Small vessel disease. Polycystic ovarian syndrome, Chronic inflammation, or Traumatic Brain Injury (TBI).

3. The method of claim 1, wherein the one or more cannabinoids is administered in a dose range of about 0.01 mg to about 600 mg.

4. The method of claim 1, wherein the one or more cannabinoids is administered via an oral, vaginal, rectal, oral mucosal, nasal, sublingual, inhalational, topical, parenteral, intravenous, intramuscular, or subcutaneous route of administration.

5. The method of claim 1, wherein the one or more cannabinoids is tetrahydrocannabinol (THC), tetrahydrocannabinolic acid (THCA), cannabidiol (CBD), cannabinol (CBN), Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM), Anandamide(AEA) and 2-Arachidonoylglycerol (2-AG), HU-210, HU-211, HU-308, HU-433, JWH-018, JWH-073, CP-47,497, JWH-200, cannabicyclohexanol, or any combination thereof.

6. The method of claim 5, wherein the one or more cannabinoids is derived from parts of a plant belonging to family Cannabaceae.

7. The method of claim 6, wherein the plant is Cannabis sativa, Cannabis indica, or Cannabis ruderalis, or hemp varieties thereof.

8. The method of claim 5, wherein the cannabinoid is THC.

9. The method of claim 8, wherein the THC is taken in a dosage of about 0.001 mg to about 0.5 mg.

10. The method of claim 8, wherein the THC is taken in a dosage of about 0.5 mg to about 2.5 mg.

11. The method of claim 8, wherein the THC is taken in a dosage of about 2.5 mg to about 10 mg.

12. The method of claim 5, wherein the cannabinoid is CBD.

13. The method of claim 12, wherein the CBD is taken in a dosage of about 0.1 mg to about 10 mg.

14. The method of claim 12, wherein the CBD is taken in a dosage of about 10 mg to about 100 mg.

15. The method of claim 12, wherein the CBD is taken in a dosage of about 100 mg to about 600 mg.

16. The method of claim 5, wherein the one or more cannabinoids is a plant extract containing said one or more cannabinoids.

17. The method of claim 1, wherein the subject is human.

18-34. (canceled)
